(19) United States
(12) Patent Application Publication
(10) Pub. No.: US 2009/0137981 A1
(43) Pub. Date: May 28, 2009

(54) METHODS OF TREATING A BLOOD VESSEL

(75) Inventor: Charles W. Kerber, La Mesa, CA (US)

Correspondence Address: KNOBBE MARTENS OLSON & BEAR LLP, 2040 MAIN STREET, FOURTEENTH FLOOR, IRVINE, CA, 92614, US

(73) Assignee: Valor Medical, San Diego, CA (US)

(21) Appl. No.: 12/276,807

(22) Filed: Nov. 24, 2008

Related U.S. Application Data
(60) Provisional application No. 60/990,209, filed on Nov. 26, 2007.

(51) Int. Cl. A61K 31/785; A61M 25/10

(52) U.S. Cl. 604/509; 424/78.35

(57) ABSTRACT

Described herein are methods for treating a blood vessel. In an embodiment, the method of treating a blood vessel comprises providing at least one manipulable tool in a blood vessel, depositing a non-solid polymerizable material into a deposition area of the vessel, wherein the polymerizable liquid hardens over time upon contact with blood in the blood vessel, and altering the shape of the polymerizable material while it hardens by manipulating the tool.

### CROSS-REFERENCE TO RELATED APPLICATIONS

This application claims priority to U.S. Provisional Patent Application Ser. No. 60/990,209, filed Nov. 26, 2007, which is hereby incorporated by reference in its entirety.

### BACKGROUND OF THE INVENTION

1. Field of the Invention

The present invention relates to methods of treating a blood vessel. The present invention further relates to depositing a polymerizable material into a blood vessel and manipulating the polymerizable material into a permanent shape to reduce turbulent blood flow within the blood vessel or to otherwise improve or alter the blood flow in a desired manner.

2. Description of the Related Art

A cerebral aneurysm is a balloon-like swelling of the wall of a blood vessel in the brain. This weakening in the wall often leads to rupture, bleeding, and death. Cerebral aneurysms are more common in elderly people, including people over 65 years old. They may be found in as high as 5% of the population. Smoking and hypertension appear to markedly increase the chance that one will develop a cerebral aneurysm. It is estimated that approximately 30,000 people in the United States are diagnosed each year with a cerebral aneurysm. However, there are an estimated 4.5 million individuals in the U.S. that have silent, undiagnosed cerebral aneurysms. This population is expected to grow with the aging of the population. Although cerebral aneurysms are among the most difficult aneurysms to treat, aneurysms can occur in other parts of the body as well, with attendant risks to the patient.

One surgical method for treating aneurysms is direct surgery. Direct surgery takes place under general anesthesia, and in the case of cerebral aneurysms, is performed by opening the skull and identifying the neck of the aneurysm, e.g., the junction between the normal blood vessel and the weakened ballooned aneurysm. After locating and exposing the aneurysm, it is isolated by applying a clip, where this is possible. However, this procedure is lengthy and requires unfavorable conditions to the patient, including opening of the skull and several days of hospitalization. Furthermore, many aneurysms are not accessible using this method.

Another method of treating an aneurysm involves endovascular surgery, which usually takes place under general anesthesia. Endovascular surgery is performed by inserting a small tube or catheter into a peripheral blood vessel (e.g., in the leg) and navigating it through the blood vessel into the aneurysm under the guidance of X-Ray. The aneurysm is then filled with tiny platinum coils injected from the small tube or catheter. Patient selection is based on the individual patient and aneurysm anatomy. An endovascular coil placement procedure can take up to 3-5 hours and require multiple, e.g. 6-12, coils to be placed in the aneurysm.

A medical device known as blood flow diverter can also be used to treat intracranial aneurysms. These blood flow diverters are formed from a porous tubular membrane and are placed in the proximity outside of an aneurysm in order to prevent blood from flowing and entering into the aneurysm. These devices can be problematic, however, because the diverter oftentimes additionally blocks the flow of blood to otherwise normal, healthy tissues. This prevents blood from reaching healthy blood vessels and restricts oxygen and other important blood-based materials from reaching other tissues. Additionally, the implantable molded devices can be prone to contamination or other bacterial activity.

More recently, compositions comprising cyanoacrylate have been used to treat aneurysms. For example, U.S. Pat. Nos. 6,037,366, 6,476,069, 6,476,070, and RE39,150, the contents of which are incorporated herein by reference in their entirety, disclose cyanoacrylate compositions which involve mixing two separate components immediately prior to administration. These “dual vial” compositions polymerize upon contact with blood and are administrated using a catheter. Dual vial compositions have been used in the past to address storage stability of the cyanoacrylate materials.

Another recent composition for treatment of aneurysms comprises a “single vial” formulation of a cyanoacrylate composition. U.S. patent application Ser. No. 12/268,318, entitled “Single Vial Formulation for Medical Grade Cyanoacrylate” (the contents of which are incorporated by reference in their entirety), discloses medical grade composition suitable for application to or in the human body, comprising a mixture of (a) a polymerizable alkyl cyanoacrylate monomer or oligomer; (b) at least one polymerization inhibitor; (c) a contrast agent; and (d) a plasticizer. The compositions described in the '318 Application polymerize in vivo, can be sealed in a single container, and are stable for more than one month at room temperature.

There exists a need for improved methods of treating blood vessels and aneurysms using compositions that polymerize when contacted with blood in the blood vessels. There further exists a need for improving the manner in which blood flows within blood vessels by manipulating a polymerizable material as it hardens upon contacting blood within the blood vessel.

### SUMMARY OF THE INVENTION

Described herein are methods for treating a blood vessel. In one embodiment, the method of treating a blood vessel comprises providing at least one manipulable tool, such as a compliant balloon, in a blood vessel, depositing a non-solid polymerizable material into a deposition area of the vessel, wherein the polymerizable liquid hardens over time upon contact with blood in the blood vessel, and altering the shape of the polymerizable material while it hardens by manipulating the manipulable tool.

In another embodiment, the method of treating a blood vessel further comprises inflating a first balloon adjacent to the deposition area prior to the depositing step, inflating a second balloon adjacent to the polymerizable material to shape the polymerizable material while it hardens, deflating the balloons, and optionally examining blood flow past the deposition area using a contrast agent.

As the polymerizable material enters the blood vessel and begins to polymerize, it can be manipulated into a permanent shape. The polymerizable material can be hardened into a solid permanent shape. Preferably, the permanent shape reduces turbulent blood flow within the blood vessel or otherwise directs or improves blood flow in a desired manner. For example, the permanent shape of the material can direct the flow of blood in the blood vessel. If the blood vessel is bifurcated or branched in any manner, the solid permanent shape can smoothly direct the flow of blood into the separate blood vessel branches.

An embodiment provides an in-situ polymerizable cyanoacrylate for treatment of a vascular aneurysm by placement of the polymerizable cyanoacrylate in a deposition area and then shaping the cyanoacrylate during polymerization to facilitate blood flow past the cyanoacrylate. Another embodiment provides use of a monomer or oligomer in the preparation of an in-situ polymerizable material for treatment of a blood vessel disorder by placement of the polymerizable material in a deposition area of a blood vessel and then shaping the material as it polymerizes using at least one manipulable tool.

In an embodiment, the polymerizable material comprises an alkyl cyanoacrylate monomer or oligomer. The polymerizable material, such as cyanoacrylate, can be shaped with at least one manipulable tool, such as a balloon. The polymerizable material described herein can be altered by at least one manipulable tool into a permanent shape that reduces turbulence of blood flow within the blood vessel. In addition, the permanent shape that the polymerizable material forms can direct the flow of blood in the blood vessel.

The polymerizable material can be used in the treatment of various blood vessel maladies. For example, the polymerizable material can be deposited into an area comprising aneurysm, such as a berry aneurysm or a saccular aneurysm. The polymerizable material can also be used to treat an arteriovenous malformation.

### BRIEF DESCRIPTION OF THE DRAWINGS

FIG. 1 illustrates an untreated blood vessel comprising an aneurysm.

FIG. 2 illustrates an initial treatment step of a blood vessel comprising an aneurysm.

FIG. 3 illustrates an intermediary treatment step of a blood vessel comprising an aneurysm.

FIG. 4 illustrates a later treatment step of a blood vessel comprising an aneurysm.

FIG. 5 illustrates a blood vessel treated according to a method of treatment described herein.

FIG. 6 illustrates a healthy, untreated bifurcated blood vessel.

### DETAILED DESCRIPTION OF THE PREFERRED EMBODIMENTS

Polymers, such as cyanoacrylates have been proposed in the past for embolization of aneurysms and for filling other spaces in the body. The present invention describes novel methods of administering polymerizable material into the body, including blood vessels or other lumens. Any known polymerizable material that is biocompatible in the human body is contemplated for use in conjunction with the methods described herein, particularly polymers that can be shaped during polymerization, do not fragment or shed during implantation or use, and are safe for long term implantation in the body. For example, both single vial formulations and dual vial formulations can be used. In an embodiment, formulations comprising a polymerizable cyanoacrylate are used. In an embodiment, a single vial formulation of an oligomer of at least one alkyl cyanoacrylate monomer, at least one inhibitor, a contrast agent, and a plasticizer is used in the methods described herein.

### Methods of Treatment

Described herein are methods for treating a blood vessel. In an embodiment, a method for treating a blood vessel comprises providing at least one compliant balloon in a blood vessel. The at least one compliant balloon can be provided in the blood vessel according to any known method, including using catheters, cannulas, syringes, micro-catheters, and the like. Multiple balloons can also be provided in the blood vessel, depending on the area to be treated. For example, one, two, three, four, or more balloons may be provided in the blood vessel. Upon administration of a non-solid polymerizable material into the blood vessel, the at least one balloon can aid in controlling the shape of the polymerizable material as it hardens. In an embodiment, the balloons are positioned in a manner that allows manipulation of a polymerizable material during its administration.

Upon positioning one or more balloons in the blood vessel, a non-solid polymerizable material is deposited into a deposition area of the vessel. As used herein, a “deposition area” means the area of the blood vessel (or other body lumen) where the polymerizable material is administered and where it hardens into a solidified mass. A deposition area can be at any location in a blood vessel or a body lumen. The positioning of the balloon(s) and the relative amount of inflation of the balloon(s) can be used to help control the size and shape of the deposition area and/or to prevent undesired migration of material during implantation. Additionally, the deposition area can also be dependent upon the ailment being treated. For example, where the area to be treated is an aneurysm, the deposition area can comprise the aneurysm, in part, or in its entirety. The deposition area can also comprise a peripheral area surrounding the location of any blood vessel disorder.

Preferably, the deposition area comprises a disorder of the blood vessel. In one embodiment, the deposition area comprises a fistula. In another embodiment, the deposition area comprises an aneurysm. Any type of aneurysm, such as saccular aneurysm, fusiform aneurysm, giant aneurysm, or traumatic aneurysm, can be treated according to the methods described herein. For example, in one embodiment, the aneurysm is a berry aneurysm. In another embodiment, the aneurysm is a saccular aneurysm. In a further embodiment, the aneurysm is an aortic aneurysm. One aspect of the disclosure involves treatment of coronary aneurysms. In another embodiment, the aneurysm is a cerebral aneurysm.

Furthermore, other blood vessel disorders, besides aneurysms, can be treated using the methods described herein. In an embodiment, the deposition area comprises an arteriovenous malformation.

Treatment, according to the methods described herein, can take place at any location in the body, including the brain. At least one balloon and polymerizable material can be guided to any part of the body using catheters, cannulas, syringes, micro-catheters, or any other similar device used for such purposes.

The non-solid polymerizable material can be administered using any suitable catheter, cannula, syringe, micro-catheter, and the like. In an embodiment, the polymerizable material is deposited with a micro-catheter. The micro-catheter can be inserted at a suitable location, such as into a blood vessel in the leg, and guided to the deposition area using known techniques, such as direct radiological visualization, preferably using radiopaque markers on the catheter, with or without injection of contrast agent. In an embodiment, the micro -catheter is advanced to the location of the deposition area, e.g. aneurysm, under direct fluoroscopic observation. In an embodiment, the micro-catheter is advanced to the location of the deposition area, e.g. aneurysm, using another visualization modality, such as MRI or ultrasound, including use of an internal ultrasound imaging system incorporated into or otherwise associated with a guiding catheter, an interventional catheter, or a guidewire.

Suitable catheters, cannulas, syringes, micro-catheters, and the like that are useful in the method steps described herein include those described in the art. For example, U.S. Pat. No. 5,795,331, entitled “Balloon Catheter for Occluding Aneurysms of Branched Vessels,” the contents of which are incorporated herein by reference, discloses a device and methods for delivering compositions. The device described combines an inflatable balloon with a catheter as a single apparatus, where the balloon is distal or proximal to the opening of the catheter. Other device examples are described in U.S. Pat. No. 5,882,334, entitled “Balloon/Delivery Catheter Assembly with Adjustable Balloon Positioning” and U.S. Pat. No. 6,015,424, entitled “Apparatus and Method For Vascular Embolization,” the contents of both references are incorporated herein by reference.

Although the disclosed procedure focuses on the use of inflatable balloons for shaping the hardening polymerizable material, it should be appreciated that any intravascular tool, such as a spatula, a grasper, or a steerable tool or a catheter itself (preferably a steerable catheter) can be used as a tool for the shaping process.

Upon administration, the polymerizable liquid hardens over time upon contact with blood in the blood vessel. The amount of time required for the polymerizable liquid to harden can vary over a wide range and depends upon a number of factors, including the composition of the polymerizable liquid and the rate at which it is administered. In an embodiment, the polymerizable material solidifies in less than a minute upon administration into the blood vessel. In one embodiment, the polymerizable material solidifies over 5 to 30 seconds following deposition (i.e., hardens to the point that it can no longer be effectively reshaped by applying external pressure). In a preferred embodiment, the polymerizable material hardens over 10 to 15 seconds.

While the polymerizable material hardens, its shape can be manipulated by altering the balloon. In an embodiment, a plurality of balloons, e.g. two, three, four, or more balloons are used to alter the shape of the polymerizable material as it hardens. In an embodiment, the balloon is manipulated to alter the polymerizable material into a permanent shape that reduces turbulence of blood flow within the blood vessel. Blood vessel disorders, such as aneurysms, frequently cause turbulent or misdirected blood flow within the blood vessel. The misdirected or turbulent blood flow can increase damage to the blood vessel walls, and eventually lead to rupture of the blood vessel.

The polymerizable material can be shaped to form smoother flow paths in the blood vessels and minimize turbulence. In an embodiment, the polymerizable material is manipulated to form improved blood flow pathways. For example, as the polymerizable material hardens, the surface of the formed solid mass can be shaped to provide a blood vessel wall that is relatively or substantially smooth. The smooth blood vessel wall allows blood to flow with minimal turbulence. One aspect of the disclosed method involves substantially reducing the amount of blood flow turbulence in the vicinity of the deposition area, compared to turbulence prior to the procedure.

In an embodiment, the polymerizable material is shaped to substantially fill a recess in the deposition area. For example, a blood vessel recess caused by an aneurysm, such as a saccular aneurysm, can be filled in with the polymerizable material. The material can be manipulated or shaped while it is being implanted and/or while it hardens using an intravascular tool to selectively apply shaping pressure to the implanted material. The intravascular tool is preferably different from the orifice from which the polymerizable material is deposited (e.g., a micro-catheter tip). One desirable end result is that the blood vessel has a smooth inner wall at the location where the aneurysm has been filled with the polymerizable material. In one embodiment, the aneurysm can be partially filled with the polymerizable material. In another embodiment, the aneurysm is completely filled with polymerizable material; indeed, it may be desirable to more than fill the aneurysm to better direct blood flow past the deposition area.

In one advantageous embodiment, the permanent shape (into which the polymerizable material is formed after deposition and while it is hardening) directs the flow of blood in the blood vessel. For example, the blood flow can be directed in any desired direction by forming a permanent shape that allows blood flow in one or more desired directions, but inhibits blood flow in another direction. This is particularly desirable where the aneurysm is located at a juncture where the blood vessel branches in two or more directions. The solid mass created by the polymerizable material can be shaped to direct the pathway of the blood flow into each branch accordingly.

In some intravascular procedures, the blood vessel comprises a dividing point, wherein the vessel divides into two or more branches, and wherein there is an aneurysm at the dividing point. Under such conditions, the polymerizable material can be formed by application of external pressure into a solid permanent shape that preferably smoothly directs the flow of blood into the separate branches. When appropriate, the deposited and shaped material divides the flow of blood evenly into the separate branches.

In another embodiment, the solid permanent shape is formed to smoothly direct the flow of blood unevenly into the separate branches. For example, it may be desirable that one of the blood vessel branches requires more blood than the other. Furthermore, it may be desirable to lessen (or in less usual circumstances to completely cut off) the flow of blood to one of the blood vessel branches. In an embodiment, the permanent shape is formed to inhibit blood flow into one branch in favor of guiding the blood flow into another branch. The blood flow can be directed in order to direct blood to certain tissues in the body.

Controlling the shape of the polymerizable material can be performed using a single tool, such as a balloon, or more than one balloon or other tool. In an embodiment, at least two balloons are used to contain and/or shape the polymerizable material. Increasing the number of balloons allows for more precise control of final shape of the material as it polymerizes. Preferably, each of the balloons can be independently inflated or deflated. As the non-solid polymerizable material is administered into the deposition area, the balloons can be pressed into it so that as it hardens into a solid mass, such that the balloons control the shape of the solid mass.

The timing of balloon inflation and deflation can vary. For example, the administrator can inflate the balloon(s) to its (their) final configuration before or after the polymerizable material administration device is in its final position. In an embodiment, the balloon(s) are each independently altered by inflation or deflation simultaneously with the administration of the polymerizable material. In an embodiment, the method of treating a blood vessel further comprises inflating at least one of the balloons prior to deposition to at least partially maintain the polymerizable in the deposition area, and then inflating a second balloon to shape the polymer.

The balloons can provide several other functions, including temporarily blocking blood flow during the course of administration of the polymerizable material. In an embodiment, blood flow is stabilized or occluded either distally or proximally to the deposition area using a temporary inflatable balloon, or a different structure having a similar function. For example, a balloon can be inflated to occlude fluid flow at points that are distal or proximal to the body space to be treated. The balloon can then be deflated and removed after a period of time once the composition has been delivered. Optionally a balloon can be juxtaposed adjacent to the body space where the composition is deposited, and inflated such that said balloon structure maintains the composition at the body space while the composition is polymerizing, and deflated for removal after some period after the composition has been delivered.

Temporary balloon occlusion stabilizes the immediate environment near the aneurysm from the disturbed flow, increased flow, turbulence, or combination thereof, created by normal unrestricted blood flow. The temporary balloon, optionally, may also be used to temporarily form a seal at the opening of the body space, while the polymerizable material that had been administered in the body space is polymerizing to its final form.

Various types of blood vessel maladies may be treated according to the methods described herein. In an embodiment, the deposition area comprises an aneurysm. FIGS. 1 through 5 illustrate an embodiment where the deposition area comprises an aneurysm located at a branched portion of a blood vessel. While the Figures provide guidance as to the principles set forth herein, the methods of treatment described herein are not limited to the depictions in FIGS. 1-5.

FIG. 1 shows an untreated blood vessel 10 comprising an aneurysm. The blood vessel 12 branches into two separate branched blood vessels 14, 16. At the dividing point of the blood vessel is an aneurysm 18. The arrows in FIG. 1 represent the blood flow within the blood vessel system. While some of the blood that flows from blood vessel 12 enters into the respective branched blood vessels 14, 16, the principal force of the flowing blood first impinges on the aneurysm 18, creating turbulence and further damaging this weakened area.

Optimal blood flow is not achieved in the illustrated embodiment due to the turbulence and disruption of the blood flow at the branch point where the aneurysm 18 is located. Additionally, the lining of the blood vessel 10 is further weakened by the strong, turbulent flow of blood into the aneurysm 18. The blood vessel lining can become so weakened at the aneurysm 18 by the blood flow that it can eventually lead to rupture of the blood vessel 10 at the aneurysm 18.

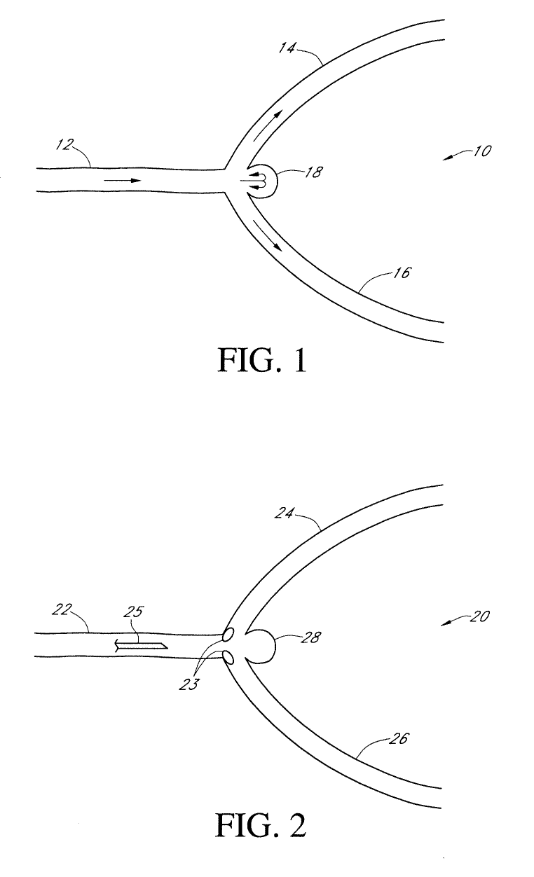

FIG. 2 illustrates an initial treatment step of a blood vessel 20 having an aneurysm 28. At least one balloon 23, (more preferably two or more balloons 23) is provided in the blood vessel 20 at a location in the vicinity of the deposition area, e.g., aneurysm 28. The balloons 23 can be guided through the blood vessel 20 to the deposition area using known methods. Preferably, the balloons 23 are put in a position to allow manipulation of the polymerizable material into a permanent shape while it is being administered into the blood vessel 20. For example, the balloons 23 can be placed at opposite sides of blood vessel 22 at the dividing point where the blood vessel bifurcates into separate branched blood vessels 24, 26. A micro-catheter 25 is guided through the blood vessel 20 to deposit the polymerizable material into the aneurysm 28.

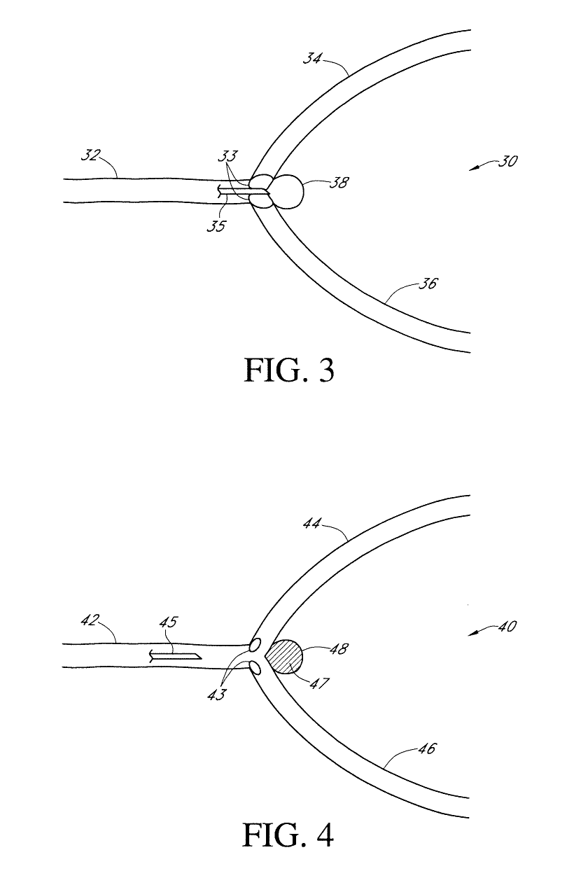

FIG. 3 illustrates one possible intermediate step in the treatment of a blood vessel 30 having an aneurysm 38. The balloons 33 are inflated to the fully form the deposition area 38 to its final configuration. The micro-catheter 35 that will deposit the polymerizable material is positioned between the balloons 33 either before or after the balloons are in their final configuration. In the embodiment represented by FIG. 3, the balloons 33 are in their final configuration before deposition of the polymerizable material from the micro-catheter. However, it is also contemplated that the balloons can be manipulated by inflation and deflation during and after deposition of the non-solid polymerization material. In an embodiment, real time shaping of the polymerizable material occurs as the polymerizable material hardens into solid mass using fluoroscopy or X-ray techniques.

In one of the disclosed methods, the balloons are each independently inflated or deflated to provide a different function. In an embodiment, at least one of the balloons 33 is inflated prior to deposition in order to at least partially maintain the polymerizable material in the deposition area. In one embodiment, at least one of the balloons 33 is inflated adjacent to the deposition area prior to the depositing step in order to control the flow of polymerizable material as it is deposited. In an embodiment, at least one of the balloons 33 is inflated adjacent to the polymerizable material to shape the polymerizable material while it hardens.

Optionally, additional balloons or other manipulable intravascular tools (not shown) can be provided in the blood vessel 32 or the separate blood vessel branches 34, 36 and inflated to temporarily inhibit the flow of blood during administration of the polymerizable material. The polymerizable material can now be deposited from the micro-catheter 35 into the deposition area 38. If the aneurysm is completely filled, the polymerizable material can take on the shape of the aneurysm at the distal end as it hardens into a solid mass. The balloons 33 can each independently be selectively inflated and/or deflated to control the shape of the proximal end of the solid mass.

FIG. 4 illustrates a later treatment step of a blood vessel 40 comprising an aneurysm after the polymerizable material has been administered. After the polymerizable material hardens into a solid mass 47 in the deposition area 48, the balloons 43 can be deflated and removed. In an embodiment, the manner in which the blood flows is examined using fluoroscopy or ultrasound techniques to confirm optimal flow of blood. For example, blood flow can be examined to confirm blow into branches 44, 46 from blood vessel 42.

If optimal flow of blood is not present, the balloons 43 can be re-positioned along with the micro-catheter 45, and further modification to the solid mass 47 can be made by further shaping with additional polymerizable material. After treatment, both the micro-catheter 45 and the balloons 43 are withdrawn after the final shape of solid mass 47 is provided. Any additional balloon (not shown), e.g. occlusion balloons used to temporarily block blood flow, in the blood vessel 42 or blood vessel branches 44, 46 can also be deflated and withdrawn.

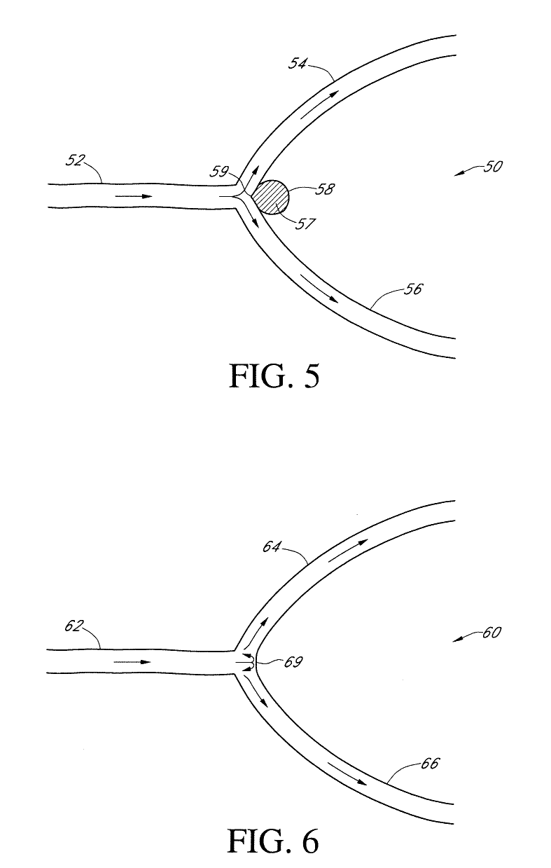

FIG. 5 illustrates a blood vessel 50 treated according to a method of treatment described herein. The arrows illustrate the flow of blood in the treated blood vessels. Blood flows much more efficiently from blood vessel 52 and then bifurcates into the blood vessel branches 54, 56. The turbulent blood flow that was present before treatment as illustrated in FIG. 1 has been significantly reduced. The polymerized solid mass 57 is shaped to substantially fill, or in some embodiments, completely fill the treated aneurysm 58 and prevent blood from flowing therein. The result is a significant decrease in the risk that the blood vessel wall will rupture. The flow of blood is directed from the blood vessel 52 evenly into blood vessel branches 54, 56 by the tip 59 of the polymerized solid mass 57. The polymerized solid mass 57 effectively provides a flow divider and directs the flow of blood in the blood vessel 50.

In the embodiment illustrated in FIG. 5, the polymerized solid mass 57 is shaped to evenly divide the flow into blood vessel branch 54 and blood vessel branch 56. The solid permanent shape of the polymerized solid mass smoothly directs the flow of blood into the separate branches. For example, the proximal end of the solid mass 57 is shaped with a tip 59, wherein the tip 59 is located approximately even with the radial center of blood vessel 52. The location of the tip 59 of the polymerized solid mass 52 can be manipulated to control the division of flow between the two blood vessel branches 54, 56.

For example, if more blood flow were desired in blood vessel branch 54 than blood vessel branch 56, then the tip 59 of the polymerized solid mass could be positioned closer to blood vessel branch 56 in order to direct the flow of blood towards blood vessel branch 54. The closer the tip 59 is to a blood vessel branch 56, the more it effectively blocks blood flow into that branch. As a result, blood flow is directed into another direction, specifically, blood vessel branch 54. In an embodiment, the polymerized solid mass is further shaped to direct the flow of blood towards one blood vessel branch over another blood vessel branch. In an embodiment, the polymerized solid mass is further shaped to completely block the flow of blood to any one or more of the branches.

FIG. 6 shows a close-up of an analogous blood vessel 60 to that shown in FIGS. 1-5. However, blood vessel 60 in FIG. 6 is a healthy, untreated blood vessel. The blood vessel 62 bifurcates at junction point 69 into two blood vessel branches 64, 66. Without a polymerized solid mass to guide the flow of blood, or divide the flow of blood, some turbulence and backflow is caused at junction point 69. In comparing the untreated, healthy blood vessel 60 of FIG. 6 to the blood vessel 50 comprising a treated aneurysm of FIG. 5, it can be seen that the methods of treatment described herein provide a blood vessel that is improved over the natural human anatomy because treated blood vessels have improved blood flow over a normal, healthy blood vessel

Another treatment step that can be used in conjunction with the methods described herein is examining the blood flow using a contrast agent and known fluoroscopy techniques. Such examination can occur during any treatment step described herein. In an embodiment, the blood flow is observed after removal of the devices from the blood vessel to examine how the blood flows past the polymerized solid mass. For example, blood flow can be observed during administration of the polymerized mass. The shape of the polymerized mass can also be observed, as well as the blood flow, using a contrast agent and known fluoroscopy techniques. Thus, real-time observation of the blood flow is possible during administration or shaping of the polymerizable material, which can aid the administrator in adjusting the polymerizable material into the appropriate shape as it solidifies.

### Polymerizable Material Compositions

Any known polymerizable material that is safe for use in the human body can be used in conjunction with the methods described herein. Preferably, the polymerizable material solidifies over the course of a short amount of time, such as less than 1 minute. In an embodiment, the polymerizable material solidifies over 5 to 30 seconds following deposition. In an embodiment, the polymerizable material solidifies over 10 to 15 seconds. In an embodiment, the polymerizable material comprises a polymerizable alkyl cyanoacrylate monomer or oligomer.

In an embodiment, the polymerizable material comprises any of the “dual vial” polymerizable compositions described in U.S. Pat. Nos. 6,037,366, 6,476,069, 6,476,070, and RE39,150, the contents of which are incorporated herein by reference in their entirety. In an embodiment, the polymerizable material comprises a “single vial” polymerizable composition described in U.S. patent application Ser. No. 12/268,318, entitled “Single Vial Formulation for Medical Grade Cyanoacrylate,” the contents of which are incorporated by reference in its entirety.

In one embodiment, the polymerizable composition comprises an oligomer of at least one alkyl cyanoacrylate monomer, at least one inhibitor, a contrast agent, and a plasticizer. For example, the alkyl cyanoacrylate monomer can be methyl cyanoacrylate, n-butyl cyanoacrylate, isobutyl cyanoacrylate, n-hexyl cyanoacrylate, 2-hexyl cyanoacrylate or 2-octyl cyanoacrylate. In one particular embodiment, the alkyl cyanoacrylate monomer comprises hexyl cyanoacrylate. In a preferred embodiment, the hexyl cyanoacrylate comprises n-hexyl cyanoacrylate.

The contrast agents used in the methods described herein can vary, and be liquid or solid. In an embodiment, the contrast agent, or opacificant, comprises a solid contrast agent, such as, gold, platinum, tantalum, titanium, tungsten and barium sulfate and the like. Preferably, the solid contrast agent can be gold suspended in an alkyl cyanoacrylate oligomer. Factors that influence the amount of opacificant can include the amount of opacificant necessary for fluoroscopic detection.

The inhibitors used in the composition can also vary. In an embodiment, the inhibitor comprises a compound selected from the group consisting of 4-methoxyphenol, 2,6-di-tert-butyl-4-methylphenol, hydroquinone, phosphoric acid, sulfur dioxide (SO₂), and any combination thereof.

A variety of plasticizers are useful in the compositions. In an embodiment, the plasticizer is selected from the group consisting of butyl benzyl phthalate, dibutyl phthalate, diethyl phthalate, dimethyl phthalate, dioctylphthalate, trialkyl acylcitrates, benzoate esters of di- and poly-hydroxy branched aliphatic compounds, tri(p-cresyl) phosphate, and any combinations thereof.

### EXAMPLES

The following examples are given to enable those of ordinary skill in the art to more clearly understand and to practice the present invention. The examples should not be considered as limiting the scope of the invention, but merely as illustrative and representative thereof.

### Example 1

### Preparation of Stabilized n-Hexyl Cyanoacrylate

Step (a) Initial Reaction

Formaldehyde frills (290 g, 9.7 moles) were added to a 3000 mL 3-necked reactor, equipped with a Dean-Stark distillation apparatus, followed by 650 mL methanol and finally 4.8 mL piperidine. The reaction mixture was stirred using an overhead stirrer and heating was initiated. The mixture was heated to between 65° C. and 80° C. and maintained in this range for 45 minutes, during which time the solution became “milky”. The temperature was reduced to ˜55° C. and n-hexyl cyanoacetate (1600 g, 8.8 moles) was slowly added. During the addition of the n-hexyl cyanoacetate, the temperature was maintained between 68° C. and 75° C. The reaction mixture color became yellowish toward the completion of the addition. An additional 100 ml methanol was used to rinse residual n-hexyl cyanoacetate into the reaction mixture via the addition funnel.

The reaction was heated to reflux and approximately 610 ml methanol was removed via Dean-Stark distillation over ˜1 hour (during which the temperature of the reaction increased from 72° C. to 78° C.) at which time the n-hexyl cyanoacrylate was formed. Subsequently, 630 ml toluene was added via an addition funnel. The mixture containing the n-hexyl cyanoacrylate was heated to remove the residual methanol and piperidine via azeotropic distillation, which occurred from 84° C. to 115° C. (uncorrected temperature). When the temperature rose to 115° C. the distillation was discontinued. The system was allowed to cool to room temperature.

Step (b) Cracking Process

The reaction apparatus was reassembled to replace the Dean-Stark distillation apparatus setup with a Vigreux distillation column. A chilled condenser with a receiver flask was attached to the distillation column. The system was set up so a vacuum could be applied as necessary. To the reaction vessel was added 50 mg polyphosphoric acid and 0.8 g 4-methoxyphenol and then the system was sealed.

The receiver flask was cooled with liquid nitrogen and then the mixture was stirred and the system placed under vacuum (5 mm Hg to 1 mm Hg). The vacuum was regulated by bleeding in argon. The reaction vessel was maintained below 150° C. and a liquid fraction containing all the added toluene was collected by distillation. The vacuum was broken using argon and then the system was blanketed with SO₂ for 3 seconds. The receiver flask containing toluene was replaced with a pre-weighed collection vessel containing 4-methoxyphenol (10 mg/100 mL vessel size, e.g. a 1 L vessel contains 100 mg of 4-methoxyphenol). The apparatus was placed under vacuum (5 mmHg to 1 mm Hg), and the reaction vessel was heated to from about 170° C. to about 190° C. (not to exceed 200° C.) to initiate cracking of the polymer, the n-hexyl cyanoacrylate monomer distills at 80° C. to 95° C. at the above stated vacuum. A forerun of 50 mL to 100 mL of n-hexyl cyanoacrylate was collected and discarded, breaking the vacuum with argon and blanketing the system with SO₂ for 3 seconds. The receiver flask containing the forerun was replaced with a pre-weighed collection vessel containing 4-methoxyphenol (10 mg/100 mL vessel size, e.g. a 1 L vessel contains 100 mg of 4-methoxyphenol). The apparatus was placed under vacuum (5 mmHg to 1 mm Hg), and the reaction vessel was heated to from about 170° C. to about 190° C. (not to exceed 200° C.) to initiate cracking of the polymer, the monomer distills at 80° C. to 95° C. at the above stated vacuum. When no further pale yellow n-hexyl cyanoacrylate monomer was collected, the heating was stopped, the vacuum was broken with argon and the system blanketed with SO₂ for 3 seconds. The rate of collection of the monomer is approximately 1 L per day, including the steps of exchanging collection vessels. Note that in the preceding process, care was taken to maintain a non-reactive atmosphere over the reaction mixture and resulting product, thus avoiding unwanted polymerization and degradation reactions. This, in turn, enhances the quality and purity of the end product, such that it is stable in a single vial formulation.

Step (c) Distilling Process

A vacuum distillation apparatus was configured with a 2 L flask (3-neck round bottom flask), magnetic stirrer, and a Vigreux column. The distillation apparatus was placed under argon and then the pale yellow n-hexyl cyanoacrylate distillate from the cracking step was added to the distillation flask. The apparatus was maintained under argon and blanketed with SO₂ for 3 seconds and stirring of the liquid in the distillation flask was initiated. The receiving flask was cooled with liquid nitrogen and then the distillation apparatus was placed under vacuum (5 mmHg to 1 mm Hg). The pale yellow n-hexyl cyanoacrylate was gradually heated with stirring until distillation initiated. Distillate was collected at a rate of one drop per minute. After ˜50 ml of forerun was collected the vacuum was broken with argon, followed by blanketing with SO₂. The forerun was discarded and a second receiving flask containing 4-methoxyphenol ((10 mg/100 mL vessel size) was placed to receive the distillate. Several fractions of distillate were collected so that the final 100 mL of distillate could be discarded. During each flask exchange the vacuum was broken with argon and the system was blanketed with SO₂. Pure n-hexyl cyanoacrylate was collected containing 4-methoxyphenol and SO₂ for use in the next step.

### Example 2

### Photochemical Viscosity Adjustment of n-hexyl Cyanoacrylate Monomer

The purified n-hexyl cyanoacrylate monomer from Example 1, containing 4-methoxyphenol, was treated with Aldrich HQ & MEHQ inhibitor remover, Sigma-Aldrich, Inc., St. Louis, Mo., USA (2005-2006 Catalog #306320), to remove the p-methoxyphenol, followed by bubbling argon through the n-hexyl cyanoacrylate monomer to remove SO₂. The viscosity of the purified n-hexyl cyanoacrylate, free of 4-methoxyphenol and SO₂, was about 4 centipoise.

The purified n-hexyl cyanoacrylate (500 g) was then introduced into an Ace glass photochemical reactor equipped medium pressure quartz mercury vapor lamp. The n-hexyl cyanoacrylate was irradiated until the liquid had a viscosity of about 20 to about 35 centipoise. The resulting oligomer material is referred to as Component A. This viscosity modification tailors the end product for use in the vasculature of a patient, with sufficiently high viscosity to allow the injected composition to remain where it is placed, in one intact mass, while at the same having a sufficiently low viscosity to allow it to be injected through a micro-catheter.

### Example 3

### Preparation of Plasticizer Component

A stock solution of tri-n-butyl O-acetylcitrate containing 4-methoxyphenol and 2,6-di-tert-butyl-4-methylphenol was prepared as follows. To tri-n-butyl O-acetylcitrate (500 grams, 1.24 mol) under argon was added 4-methoxyphenol (750 PPM) and 2,6-di-tert-butyl-4-methylphenol (750 PPM). The mixture was stirred until homogeneous. Sulfur dioxide (SO₂, 600 PPM) was bubbled through the tri-n-butyl O-acetylcitrate solution containing 4-methoxyphenol and 2,6-di-tert-butyl-4-methylphenol. The resulting material is referred to as Component B.

### Example 4

### Component C: Formulation of Component A with Component B

The UV treated n-hexyl cyanoacrylate (Component A, 500 g) was combined with Component B (250 g) at room temperature and mixed until homogeneous. The viscosity of the resulting product was from about 20 to about 35 centipoise. The above combination of Component A and Component B affords Component C.

### Example 5

### Preparation of Single Vial Formulation

Component C (1.5 mL) is added to a 5 mL vial containing fine mesh gold (0.9 g,) and the vial is placed under argon. The vial is then sealed and heat sterilized. The single vial formulation is stable for over 1 year.

### Example 6

### Preparation of 2-hexyl Cyanoacrylate

This prospective procedure is based on procedures developed employed for preparing n-hexyl cyanoacrylate, as is taught in the preceding example.

Equip a 5 liter three-necked flask with a reflux condenser, Dean-Stark trap, an addition funnel and a mechanical stirrer with a glass paddle in a 5 liter heating mantle. To the flask is added the following components, prills of paraformaldehyde (136 g, 4.5 moles), methanol (300 mL) and pyridine (2.2 mL). The reaction mixture is stirred and heated to between 65° C. and 80° C. for 45 min. The heating is cooled to ˜55° C. and 2-hexyl cyanoacetate (736 g, 4.1 moles)is added drop wise via an addition funnel. The reaction is exothermic and the rate of addition should be adjusted to keep the reaction mixture temperature between 68° C. and 75° C. An additional 46 mL of methanol is used to rinse the addition funnel. Collect the methanol distilled from the reaction flask through the Dean-Stark trap. Measure the amount recovered. Continue the distillation until 80% or more of the original volume of methanol is recovered over a one hour period of time. Subsequently, toluene (290 mL) is added via the addition funnel. The mixture is heated to remove the residual methanol and piperidine via azeotropic distillation, the distillation occurs from 84° C. to 115° C. (uncorrected temperature). When the temperature reaches 115° C. the distillation is discontinued. The system is allowed to cool to room temperature before reaction apparatus is reassembled for the next step

The reaction apparatus is reassembled to replace the Dean-Stark distillation apparatus setup with a Vigreux distillation column to which a chilled condenser was attached and a receiver flask. The system is set up so a vacuum can be applied as necessary. To the reaction vessel is added polyphosphoric acid (23 mg) and 4-methoxyphenol (0.37 g) and then the system is sealed.

The receiver flask was cooled with liquid nitrogen and then the mixture was stirred and the system placed under vacuum (5 mmHg to 1 mm Hg). The vacuum is regulated by bleeding in argon. The reaction vessel is maintained below 150° C. and a liquid fraction containing remaining toluene is collected. The applied vacuum is isolated from the system and the vacuum is broken with argon. Subsequently, the system is blanketed under SO₂ for 3 seconds.

The vacuum is broken using argon and then the system was placed under SO₂ for 3 seconds. The collection vessel containing distillate is replaced with a pre-weighed collection vessel containing 4-methoxyphenol (10 mg/100 mL vessel size, e.g. a 1 L vessel contains 100 mg of 4-methoxy phenol). The reaction apparatus is placed under vacuum (0.1-0.5 mm Hg), and the reaction vessel is heated to from about 170° C. to about 190° C. (not to exceed 200° C.) to initiate cracking of the polymer. A forerun of 50 mL to 100 mL of 2-hexyl cyanoacrylate was collected and discarded, breaking the vacuum with argon and blanketing the system with SO₂ for 3 seconds. The receiver flask containing the forerun was replaced with a pre-weighed collection vessel containing 4-methoxyphenol (10 mg/100 mL vessel size, e.g. a 1 L vessel contains 100 mg of 4-methoxyphenol). The apparatus was placed under vacuum (5 mmHg to 1 mm Hg), and the reaction vessel was heated to from about 170° C. to about 190° C. (not to exceed 200° C.) to initiate cracking of the polymer, the monomer distills at 80° C. to 95° C. at the above stated vacuum. The collection vessel containing 2-hexyl cyanoacrylate monomer is replaced with another empty pre-weighed collection vessel containing 4-methoxyphenol (10 mg/100 mL vessel size) and the above process is repeated until the majority of the 2-hexyl cyanoacrylate monomer is collected (blanket with sulfur dioxide at each flask exchange). The rate of collection of the monomer is 1 L per day, including the steps of exchanging collection vessels.

### Example 7

### Preparation of n-Pentyl Cyanoacrylate

This prospective procedure is based on procedures developed employed for preparing n-hexyl cyanoacrylate, as is taught in the preceding examples.

Equip a 10 liter three-necked flask with a reflux condenser, Dean-Stark trap, an addition funnel and a mechanical stirrer. To the flask is added the following components, prills of paraformaldehyde (272 g, 9 moles), methanol (600 mL) and pyridine (4.4 mL). The reaction mixture is stirred and heated to between 65° C. and 80° C. for 45 min. The heating is removed and the mixture cools to ˜55° C. and then n-pentyl cyanoacetate (1372 g, 8.2 moles) is added drop wise via an addition funnel. The reaction is exothermic and the rate of addition should be adjusted to keep the reaction mixture temperature between 68° C. and 75° C. An additional 92 mL of methanol is used to rinse the addition funnel. The methanol is distilled from the reaction flask through the Dean-Stark trap and collected. The distillation is continued until 80% or more of the original volume of methanol is recovered over a one hour period of time. Subsequently, toluene (580 mL) was added via the addition funnel. The mixture is heated to remove the residual methanol and piperidine via azeotropic distillation, the distillation occurs from 84° C. to 115° C. (uncorrected temperature). When the temperature reaches 115° C. the distillation is discontinued. The system is allowed to cool to room temperature before reaction apparatus is reassembled for the next step

The reaction apparatus is reassembled to replace the Dean-Stark distillation apparatus setup with a Vigreux distillation column to which a chilled condenser was attached and a receiver flask. The system is set up so a vacuum can be applied as necessary. To the reaction vessel is added polyphosphoric acid (46 mg) and 4-methoxyphenol (0.74 g) and then the system is sealed.

The receiver flask is cooled with liquid nitrogen and then the mixture is stirred and the system is placed under vacuum (5 mmHg to 1 mm Hg). The vacuum is regulated by bleeding in argon. The reaction vessel is maintained below 150° C. and a liquid fraction containing remaining toluene is collected. The applied vacuum is isolated from the system and the vacuum is broken with argon. Subsequently, the system is blanketed under SO₂ for 3 seconds.

The vacuum is broken using argon and then the system was placed under SO₂ for 3 seconds. The collection vessel containing distillate is replaced with a pre-weighed collection vessel containing 4-methoxyphenol (10 mg/100 mL vessel size, e.g. a 1 L vessel contains 100 mg of 4-methoxy phenol). The reaction apparatus is placed under vacuum (0.1-0.5 mm Hg), and the reaction vessel is heated to from about 170° C. to about 190° C. (not to exceed 200° C.) to initiate cracking of the polymer. A forerun of 50 mL to 100 mL of n-pentyl cyanoacrylate was collected and discarded, breaking the vacuum with argon and blanketing the system with SO₂ for 3 seconds. The receiver flask containing the forerun was replaced with a pre-weighed collection vessel containing 4-methoxyphenol (10 mg/100 mL vessel size, e.g. a 1 L vessel contains 100 mg of 4-methoxyphenol). The apparatus was placed under vacuum (5 mmHg to 1 mm Hg), and the reaction vessel was heated to from about 170° C. to about 190° C. (not to exceed 200° C.) to initiate cracking of the polymer, the monomer distills at 80° C. to 95° C. at the above stated vacuum. The collection vessel containing n-pentyl cyanoacrylate monomer is replaced with another empty pre-weighed collection vessel containing 4-methoxyphenol (10 mg/100 mL vessel size) and the above process is repeated until the majority of the n-Pentyl cyanoacrylate monomer is collected (blanket with sulfur dioxide at each flask exchange). The rate of collection of the monomer is 1 L per day, including the steps of exchanging collection vessels.

All references cited herein are incorporated herein by reference in their entirety. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the preferred embodiments. At the very least, and not as an attempt to limit the application of the doctrine of equivalents to the scope of the claims, each numerical parameter should be construed in light of the number of significant digits and ordinary rounding approaches.

The above description discloses several methods and materials of the preferred embodiments. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims.

What is claimed is:

1. A method for treating a blood vessel, comprising:
providing at least one manipulable tool in a blood vessel;
depositing a non-solid polymerizable material into a deposition area of the vessel, wherein the polymerizable liquid hardens over time upon contact with blood in the blood vessel; and thereafter
altering the shape of the polymerizable material while it hardens by manipulating the tool.

2. The method of claim 1, wherein the tool is a balloon.

3. The method of claim 1, wherein the tool is manipulated to alter the polymerizable material into a permanent shape that reduces turbulence of blood flow within the blood vessel.

4. The method of claim 1, wherein the polymerizable material is shaped to substantially fill a recess in the deposition area.

5. The method of claim 1, wherein the polymerizable material comprises a polymerizable alkyl cyanoacrylate monomer or oligomer.

6. The method of claim 1, wherein the polymerizable material solidifies over 5 to 30 seconds following deposition.

7. The method of claim 1, wherein the polymerizable material solidifies over 10 to 15 seconds.

8. The method of claim 3, wherein the permanent shape directs the flow of blood in the blood vessel.

9. The method of claim 3, wherein the blood vessel comprises a dividing point, wherein the vessel divides into two or more branches, and wherein the dividing point comprises an aneurysm.

10. The method of claim 9, wherein the solid permanent shape smoothly directs the flow of blood into the separate branches.

11. The method of claim 9, wherein the polymerizable material is deposited with a micro-catheter, and wherein the tool is separate from the micro-catheter.

12. The method claim 1, wherein at least two balloons are used to contain and/or shape the polymerizable material.

13. The method of claim 12, further comprising:
inflating at least one of the balloons prior to deposition to at least partially maintain the polymerizable in the deposition area.

14. The method of claim 1, wherein the deposition area comprises an aneurysm.

15. The method of claim 14, wherein the aneurysm is a berry aneurysm.

16. The method of claim 14, wherein the aneurysm is a saccular aneurysm.

17. The method of claim 1, wherein the deposition area comprises an arteriovenous malformation.

18. The method of claim 1, further comprising:
inflating a first balloon adjacent to the deposition area prior to the depositing step;
inflating a second balloon adjacent to the polymerizable material to shape the polymerizable material while it hardens;
deflating the balloons; and
examining blood flow past the deposition area using a contrast agent.
